(19) United States
(12) Patent Application Publication
(10) Pub. No.: US 2019/0262006 A1
(43) Pub. Date: Aug. 29, 2019

(54) SURGICAL BUR AND RELATED SURGICAL INSTRUMENTS

(71) Applicant: Medtronic Xomed, Inc., Jacksonville, FL (US)

(72) Inventors: Jeffrey M. Schwamb, Saint Marys, GA (US); Phillip J. Berman, Jacksonville, FL (US); John R. Prisco, Jacksonville, FL (US)

(73) Assignee: Medtronic Xomed, Inc., Jacksonville, FL (US)

(21) Appl. No.: 15/908,029

(22) Filed: Feb. 28, 2018

(51) Int. Cl. A61B 17/16; A61B 17/32

(52) U.S. Cl. CPC A61B 17/1615; A61B 17/1688; A61B 17/32002; A61B 2017/320004; A61B 2217/007; A61B 2217/005; A61B 17/1633

(57) ABSTRACT

A surgical cutting instrument including an outer tubular member having a proximal section, an intermediate section and a central lumen. An inner tubular member is rotatably received within the central lumen and includes a distal end having a bur extending distally beyond, and exposed relative to, the distal section of the outer tubular member. The bur defines an aspiration pathway that is at least partially oriented at an angle with respect to a central axis of the bur. The aspiration pathway terminates at an opening that can be provided through a head of the bur or proximal to the head. Various embodiments of the disclosure have been shown to reduce clogging of the aspiration pathway during use, particularly during end on drilling. Various embodiments further include a sleeve for reducing lumen clogging as well as irrigation pathways that increase cooling of embodiments utilizing a distal bearing assembly.

### BACKGROUND

Powered surgical instruments have been developed for use in many orthopedic ear-nose-throat (ENT) operations as well as other operations in and around the skull. One type of cutting instrument includes a bur supported by an inner tubular member that is rotatable with respect to an outer tubular member. The bur is used to debride a target bone of a treatment site. In many instances, the bur and/or treatment site are irrigated to facilitate lubrication of the treatment site as well as to cool the bur. In other instances, aspiration is applied to the treatment site to remove debrided bone as well as to remove excess fluid. The present disclosure addresses problems and limitations associated with the related art.

### SUMMARY

The present inventors have discovered that with many surgical devices targeted to sinus and skull based surgeries, for example, frequent clogging of a suction or aspiration path in a bur of the surgical device occurs, which increases the procedure time and patient risk. In sinus and skull based surgeries, end-on drilling is often required, which is aligned with a centralized suction path of the bur. The present inventors recognized that end-on drilling packs bone and tissue debris direction into the centralized suction path, forcing the surgeon to remove the bur from the surgical instrument and clear the clog prior to proceeding. The present inventors have discovered that this problem is further pronounced when drilling on bone such as the clivus as this type of cancellous bone presents a greater propensity to clogging and requires almost constant end-on drilling throughout the procedure.

Various disclosed embodiments provide a system including a surgical cutting instrument that has an outer tubular member having a proximal section, an intermediate section and a central lumen. An inner tubular member is rotatably received within the central lumen and includes a distal end forming a bur extending distally beyond, and exposed relative to, the distal section of the outer tubular member. The bur includes an aspiration pathway at least part of which is oriented at an angle with respect to a central axis of the bur, which reduces clogging of the aspiration pathway. Various embodiments also include a sleeve positioned within the inner tubular member that reduces friction as the debris is removed and transported through the instrument.

In some embodiments, a bearing assembly is coupled to the outer tubular member and the inner tubular member to inhibit axial movement of the bur. Optionally, an inner portion of the outer tubular member is configured to include a plurality of elongate recessed surfaces, at least some of which have an aperture that is aligned with the placement of a ball of the bearing assembly. The instrument can be configured such that irrigation fluid can be directed along the elongate recessed surfaces, through the apertures and to the balls to cool the balls and bearing assembly during use of the instrument.

### BRIEF DESCRIPTION OF THE DRAWINGS

FIG. 1 is perspective view of a system including a surgical debriding instrument having a bur, in accordance with principles of the present disclosure (some system elements are shown schematically for ease of illustration).

FIG. 2 is an exploded view of select components of the instrument.

FIG. 3A is an enlarged, partially-exploded view of a distal end of the surgical instrument of FIGS. 1-2.

FIG. 3B is an assembled view of the distal end of the surgical instrument of FIGS. 1-3A.

FIGS. 4A-4C collectively illustrate cross-sectional views of the surgical instrument of FIG. 1.

FIG. 5 is a cross-sectional view of the bur of FIGS. 1-4B.

FIG. 6 is a cross-sectional view of an alternate bur.

FIG. 7A is a side view of yet another bur.

FIG. 7B is a cross-sectional view of the bur of FIG. 7A.

### DETAILED DESCRIPTION

Surgical systems and instruments embodying principles of the present disclosure can be employed in various types of surgery including, but not limited to, various sinus procedures, skull base tumor removal (such as pituitary tumors, clivus chordomas, etc.), mastoidectomy, temporal bone tumor removal, craniotomy, a modified Lothrop procedure, spinal diseases, notchplasty, acromioplasty, laminotomy, laminectomy and the like.

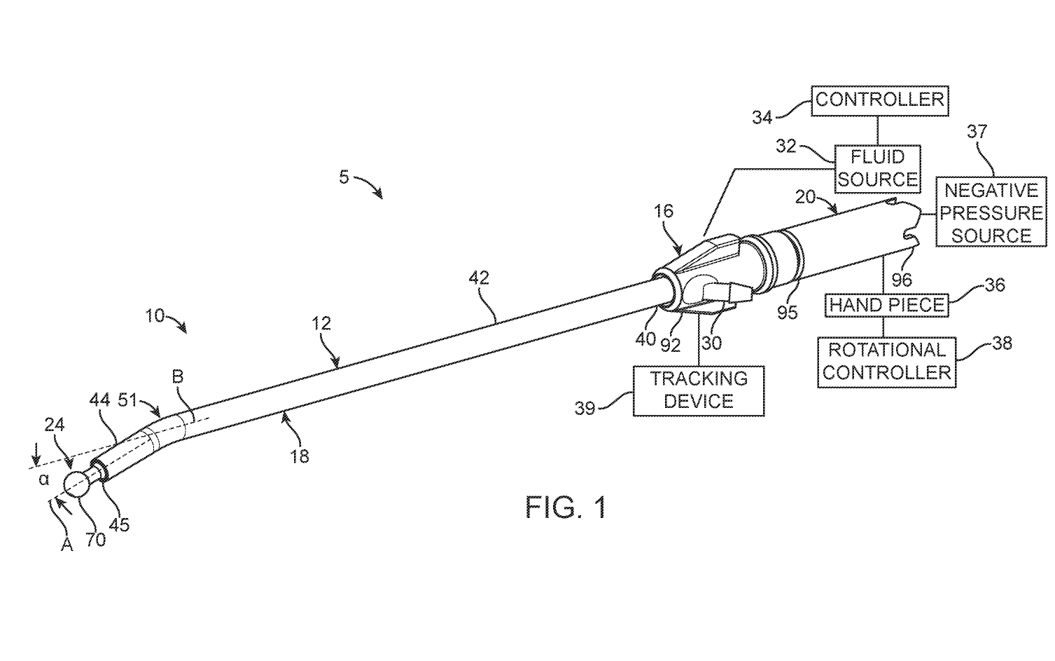

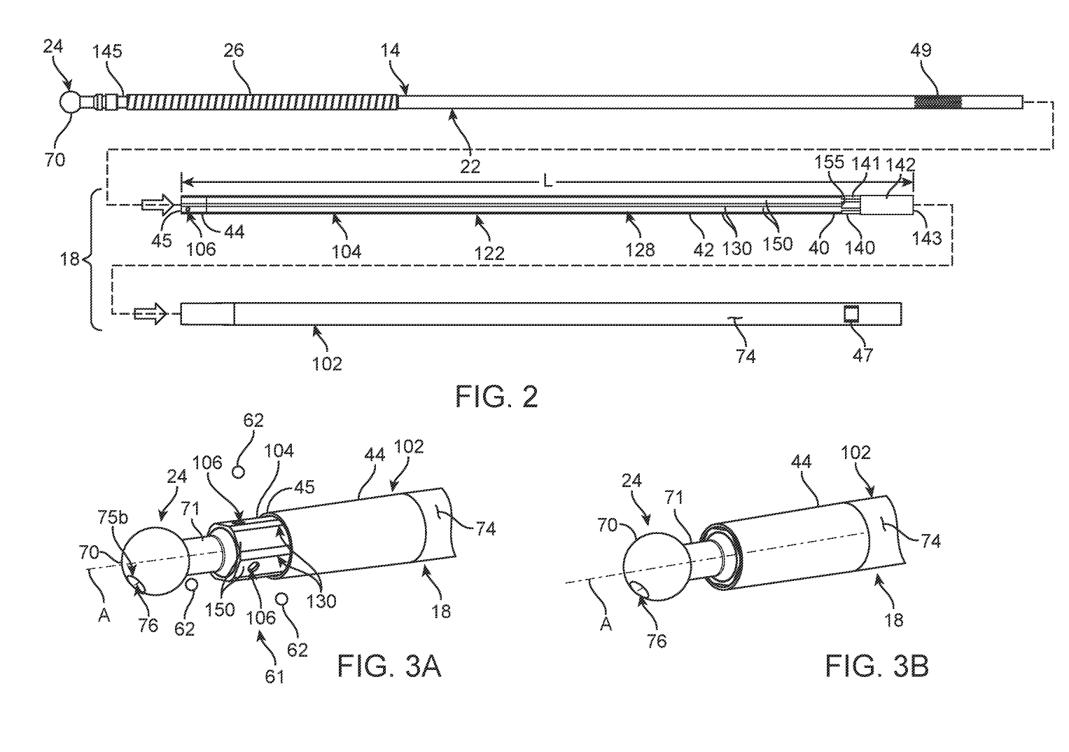

FIG. 1 illustrates a system 5 including a surgical micro-burring instrument 10, which is collectively illustrated in FIGS. 1-5. To summarize, the instrument 10 includes an outer tubular assembly 12 and an inner tubular assembly 14 coupled to a bur 24, each of which are individually described in greater detail below. With particular reference to FIGS. 1-2, the outer tubular assembly 12 generally includes an outer hub 16 and an outer tubular member 18, whereas the inner tubular assembly 14 generally includes an inner hub 20 and an inner tubular member 22. A bur 24 is provided at a distal end of the inner tubular member 22. As best illustrated in FIG. 1, the outer tubular member 18 extends distally from the outer hub 16. To this end, the outer hub 16 can assume a wide variety of forms. In some embodiments, the outer hub 16 comprises an irrigation port 30 configured for fluid communication via tubing (not shown) with a fluid source 32 controlled by a controller 34. The outer tubular member 18 and the inner tubular member 22 are formed from biocompatible metallic materials, such as stainless steel, titanium alloys, and the like. Accordingly, at least the outer tubular member 18 defines a generally rigid member.

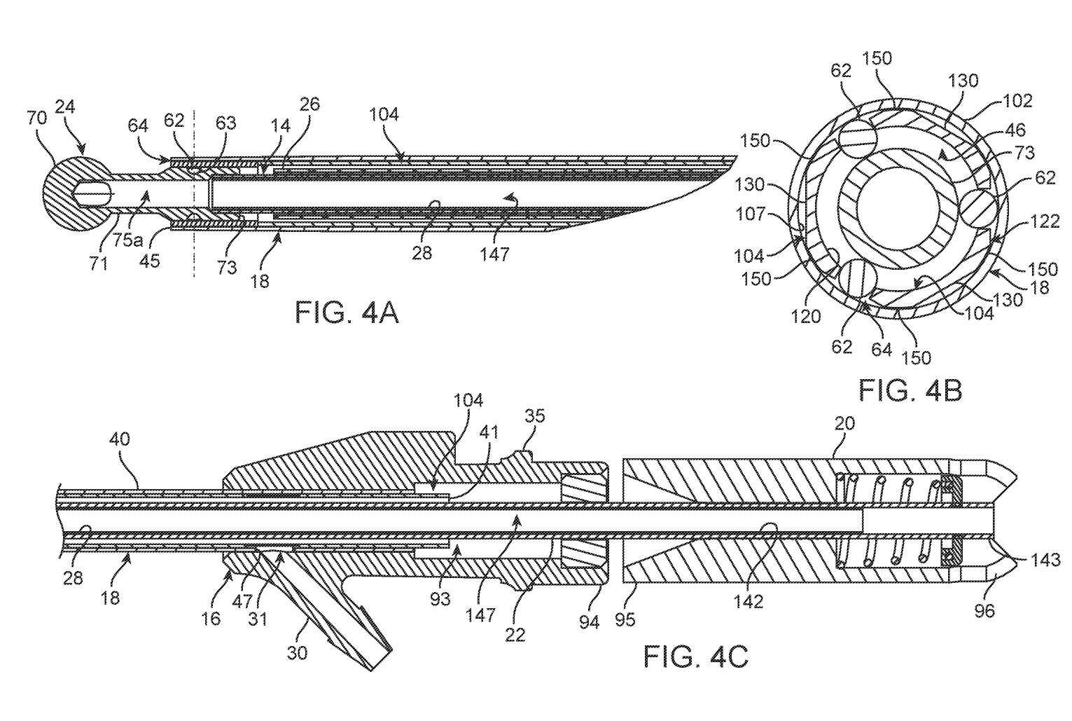

The micro-burring instrument 10 can be assembled by coaxially positioning the inner tubular member 22 within the outer tubular member 18. With particular reference to FIGS. 1 and 4C, the inner hub 20 (at distal end 95 of inner hub 20) abuts against the outer hub 16 (at a proximal end 94 of the outer hub 16). With this in mind, the inner tubular member 22 and inner hub 20 of inner tubular assembly 14 is rotatable relative to the outer tubular member 18 and the outer hub 16 of the outer tubular assembly 12. To this end, a distance of separation between the inner hub 20 and the bur 24 is greater than a distance of separation between the outer hub 16 and a distal end 45 of outer tubular member 18, thereby dictating that a desired position of the bur 24 will be exposed relative to the outer tubular member 18. In particular, the inner tubular member 22 is coaxially disposed within the outer tubular member 18 such that the distal end 45 of the outer tubular member 18 is proximal to the bur 24 (or any alternate bur disclosed herein) and to a distal section 145 of inner tubular member 22. The outer hub 16 can further define a shoulder 35 for engaging a handpiece 36.

The bur 24 is generally a solid member that can assume a variety of forms and is adapted with an abrasive or rough surface to cut or abrade bone upon rotation thereof. In some embodiments, the bur 24 includes a head 70 forming a cutting surface and a body 71 extending proximally from the head 70. While the head 70 of this illustrated embodiment is shown to have a spherical configuration, it will be appreciated that other configurations can be used including, but not limited to, cylindrical, hemispherical, ellipsoidal, fluted and pear-shaped configurations.

A central lumen 147 of the inner tubular member 22 is coupled to a negative pressure source 37 of the system 5 that provides suction to the bur 24 via the central lumen 147. In this embodiment, the central lumen 147 is connected to an aspiration pathway 75a, 75b provided within the bur 24. The central lumen 147 serves as an aspiration conduit for the micro-burring instrument 10. When the aspiration pathway 75a, 75b and central lumen 147 are applied to treat a target site, the aspiration pathway 75a, 75b extending through bur 24 enables periodic or continuous aspiration via the central lumen 147 of the inner tubular member 22 to remove abraded bone or tissue from a target site.

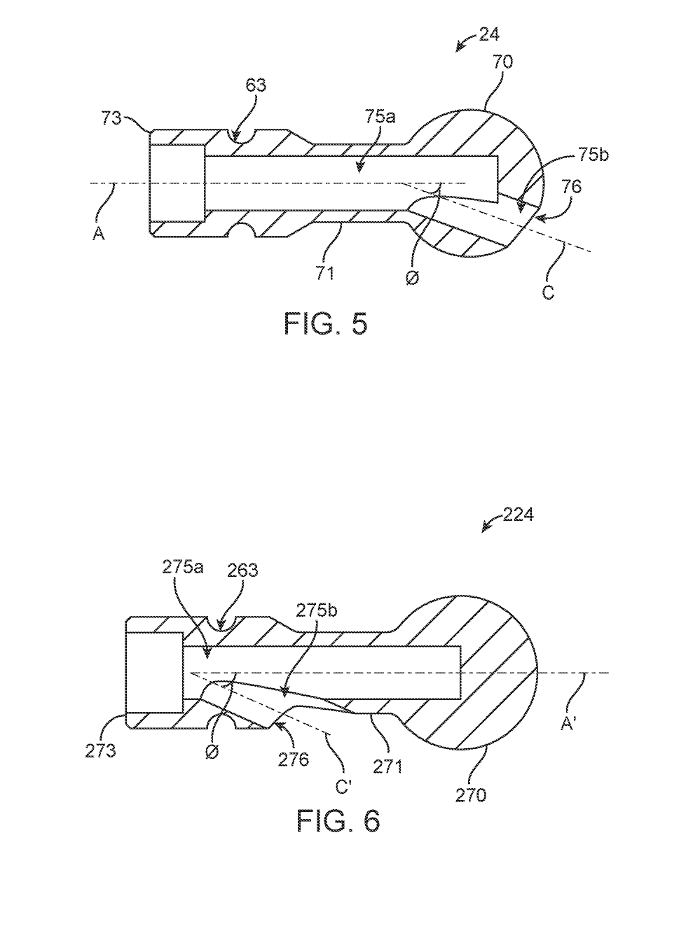

The aspiration pathway may be described as including two portions designated as 75a, 75b. The first portion of the aspiration pathway 75a interconnects the central lumen 147 and the second aspiration pathway portion 75b. As shown in FIG. 4A, the first portion 75a can be formed within the bur 24 so that it can be aligned with the central lumen 147 to form a continuous aspiration path that can optionally be linear. As is best illustrated in FIG. 5, the second portion of the aspiration pathway 75b is configured to have a centerline C that is not aligned with (i.e. not the same as) a central axis A of the body 71 and the bur 24, which reduces clogging of the aspiration pathway 75a, 75b. Because the first portion of the aspiration pathway 75a shares a centerline with the central axis A of the bur 24, the centerline of the second portion of the aspiration pathway 75b is also not aligned with the centerline of the first portion of the aspiration pathway 75a. In other words, the centerline of the first portion of the aspiration pathway 75a is also represented as A in FIG. 5. It is noted, however, that the centerline of the first portion of the aspiration pathway 75a need not be the same as the central axis A. In some embodiments, the centerline C of the second portion of the aspiration pathway 75b is oriented at an angle ϕ of about 10 to about 40 degrees with respect to the central axis A. In some embodiments, the angle ϕ is orientated at an angle of about 18 to about 22 degrees with respect to the central axis A. In the illustrated embodiment, the angle ϕ is about 20 degrees with respect to the central axis A. The present inventors discovered that the angle ϕ is important not only for achieving suction of debris without clogging, but also to allow the clearing of any potential clogs of the aspiration pathway 75a, 75b with a stylet or the like (not shown). If the angle ϕ is too large, the stylet or other tool used to clear any clogs will likely not pass through the cutting member to effectively clear the clog.

As previously described, the head 70 of the bur 24 forms the aspiration pathway or lumen 75a, 75b that extends through the body 71 of the bur 24 and which is open to the central lumen 147 defined by inner tubular member 22. By forming the aspiration pathway 75a, 75b to extend through bur 24, the surgical instrument 10 has a smaller cross-sectional profile as compared to an instrument in which an aspiration conduit is provided external to the bur. With this in mind, this smaller cross-sectional profile provides instrument 10 with greater maneuverability to enable distal section 44 of instrument 10 to pass through various soft tissues and bony structures with less likelihood of the instrument 10 catching on soft tissues and bony structures encountered along a path to a treatment site at which rotation of bur 24 is deployed.

Referring in particular to FIGS. 1, 2 and 4A-4C, the inner tubular member 22, which can be used with all surgical instruments and burs of the disclosure, is configured to extend from the inner hub 20, which is configured for selective attachment to the handpiece 36 (FIG. 1) that can be operated to automatically rotate the inner tubular member 22 during use. In particular, as best shown in FIG. 4C, the inner hub 20 extends from the distal end 95 to the proximal end 96 and is configured to be engaged by the handpiece 36 for handling the instrument 10. In one particular example, a rotational controller 38 (via a connection between handpiece 36 and inner hub 20) enables selective rotational control over inner tubular member 22 to cause high-speed rotation of the bur 24 for debriding or otherwise cutting a target bone or tissue.

The inner tubular member 22 is sized to be coaxially received within the outer tubular member 18 and receives a proximal end 73 of the bur 24. The inner tubular member 22 includes a proximal section (located at a non-recess portion 142) with proximal end 143 and distal section 145. The inner tubular member 22 can be flexible and additionally comprise a spring section 26 positioned proximal to bur 24 at the distal section 145. The spring section 26 imparts flexibility into the inner tubular member 22 such that the inner tubular member 22 can assume a curvature of the outer tubular member 18, if applicable.

In one embodiment, an inner surface of inner tubular member 22 defines at least a portion or all of the lumen 147. In some embodiments, the inner tubular member 22 can include an inner sleeve 28 (FIGS. 4A-4B), which may define all or at least part of the lumen 147. The inner sleeve 28 can be made of Vestamid® ML18 or the like, which has a low coefficient of friction (about 0.0200) so that debris transported through the sleeve 28 has less tendency to clog within the instrument 10 (Vestamid® ML18 is available from Evonik Industries Corp. of Essen, Germany). In some embodiments, the inner tubular member 22 additionally comprises a knurled portion 49 (shown only in FIG. 2). In one aspect, the knurled portion 49 facilitates securing the inner tubular member 22 to an inner portion of outer hub 16.

The outer tubular member 18 can generally be described as an elongated tubular body defining a proximal section 40 with proximal end 41 (FIG. 4C), an intermediate section 42, a distal section 44 with distal end 45 (FIG. 2), and a central lumen 46. The outer tubular member 18 can optionally define a slight bend, as referenced generally by 51, at a junction between distal section 44 and proximal section 40 of the instrument 10. In one embodiment and particular reference to FIG. 1, the bend 51 is configured to cause the central axis (as represented by the dashed line A) of the bur 24 (which is also a central axis of the distal section 44) to define an angle α in the range of about 10° to about 90°, relative to a central axis (as represented by dashed line B) of the proximal section 40 of the instrument 10. Among other uses, this bend 51 is particularly useful to facilitate proper positioning the distal section 44 during a skull-based procedure, among other surgical procedures favoring the bend 51 in the distal section 44. Alternatively, other constructions can be employed. For example, the bend 51 can be eliminated such that outer tubular member 18 is substantially straight.

The central lumen 46 extends from the proximal section 40 to the distal section 44. In this regard, the distal section 44 is open at the distal end 45 thereof to enable the inner tubular member 22 to extend distally beyond the distal end 45 of outer tubular member 18. Similarly, the proximal section 40 is open at a proximal end 41 thereof to facilitate positioning of the inner tubular member 22 within the central lumen 46. Moreover, the proximal section 40 comprises a proximal window 47 located distally of proximal end 41.

In one suitable configuration, as is best illustrated in FIG. 4C, the proximal section 40 is inserted into a lumen 93 of the outer hub 16 to secure the outer tubular member 18 to the distal section 92 of the outer hub 16. The proximal section 40 is advanced proximally within the lumen 93 of the outer hub 16 until the window 47 is aligned underneath the bottom opening 31 of the irrigation port 30, and then secured in this position to maintain fluid communication between the irrigation port 30 and the proximal window 47. In addition, in this configuration, proximal end 41 is open to the lumen 93 of the outer hub 16. Accordingly, in one aspect of the disclosure, the proximal section 40 has an outer diameter adapted to receive the outer hub 16 thereon. The remainder of the outer tubular member 18, in some embodiments, provides a relatively uniform outer diameter selected to perform the desired sinus procedure and a relatively uniform inner diameter selected to rotatably receive the inner tubular member 22. For example, in one embodiment, the intermediate section 42, as well as the distal section 44, permit use of the inner tubular member 22/bur 24 as part of a sinus procedure.

In one example embodiment, the outer tubular member 18 generally includes an outer portion 102 and an inner portion 104. The outer portion 102 of the outer tubular member 18 defines a hollow sleeve. The outer portion 102 has an inner diameter sized and adapted to receive inner portion 104. The outer portion 102 also defines an outer surface 74 of the outer tubular member 18 and provides a generally uniform and generally smooth outer diameter. Referring in particular to FIGS. 4A-4C, while the inner portion 104 can take many forms, in one configuration the inner portion 104 defines an inner surface 120 and an outer surface 122. The inner surface 120 defines a generally uniform diameter and is generally uniformly smooth from the proximal section 40, through the intermediate section 42, to the distal section 44. However, the outer surface 122 defines an array of elongate recessed surfaces 130 extending from the distal section 44, along intermediate section 42, and through at least a portion of proximal section 40. In one embodiment, the elongate recessed surfaces 130 extend along a majority of a length L of inner portion 104 (and therefore a majority of a length of outer tubular member 18) before terminating adjacent a circular recess 140 (FIG. 2) that extends transversely to the elongate recessed surfaces 130. In one aspect, the circular recess 140 forms a ring extending about a circumference of the outer surface of the inner portion 104. The circular recess 140 is in fluid communication simultaneously with each of the elongate recessed surfaces 130. Each elongate recessed surface 130 is separated by the protrusions 150.

While a variety of techniques may be used to form the inner portion 104, in one embodiment, the inner portion 104 is formed by providing a generally tubular sleeve (not shown) having a first thickness and then cutting an outer surface of the sleeve (corresponding to outer surface 122) to create each elongate recess surface 130. Accordingly, with reference to FIG. 4B, the protrusions 150 generally define the original, first thickness of the sleeve while the elongate recessed surfaces 130 extending between the respective protrusions 150 comprise a second thickness substantially less than the first thickness. The difference between the first thickness and the second thickness will then define a height of the elongate recessed surfaces 130. In one aspect, the height of each elongate recessed surface 130, the width of each elongate recessed surface 130, and the number of recesses defines the cross-sectional area, an interior passage 64, which is available to optionally send irrigation fluid.

As illustrated in FIG. 2, in one aspect, the outer surface 122 of the inner portion 104 further defines the non-recess portion 142 proximal to circular recess 140. This non-recess portion 142 is sized and adapted to be sealingly secured to an inner surface 107 of outer portion 102. In one embodiment, non-recess portion 142 is laser welded relative to the inner surface 107 of outer portion 102. This arrangement secures the inner portion 104 to outer portion 102 at proximal section 40 of the outer tubular member 18 while simultaneously defining a terminal end of the fluid communication and interior passage 64. Accordingly, fluid flowing into the outer tubular member 18 at proximal section 40 (from the irrigation port 30 and fluid source 32) will enter through proximal window 47 of outer tubular member 18, and flow through circular recess 140 just distal to non-recess portion 142 of inner portion 104 before proceeding along the elongate recessed surfaces 130 via interior passages 64, as discussed in further detail below.

As best seen in FIGS. 2 and 4B, the elongate recessed surfaces 130 of the inner portion 104 (of outer tubular member 18) form an array 128 of elongate recessed surfaces 130 uniformly spaced apart about the circumference of inner portion 104 with each elongate recessed surface 130 being defined between an adjacent pair of raised protrusions 150 formed on the outer surface 122 of the inner portion 104. In the one example configuration, the array 128 includes six elongate recessed surfaces 130 that are spaced apart uniformly (i.e., equidistant or substantially equidistant from each other) about the circumference of the outer surface 122 of the inner portion 104. In other envisioned configurations, there can be greater or fewer than six elongate recessed surfaces 130. Nevertheless, at least one elongate recessed surface 130 is provided to form at least one interior passage 64 of the outer tubular member 18 (only one interior passage 64 is labeled in FIG. 4B for ease of illustration, however, it can be seen that six interior passages are provided, one for each elongate recessed surface 130). Configurations with a greater number of elongate recessed surfaces 130 (as compared to fewer elongate recessed surfaces) spaced apart uniformly about the circumference of the inner portion (and consequently about the circumference of the outer tubular member 18) provide more balance to the fluid flow over the elongate recessed surfaces 130 and through the interior passages 64. This arrangement enables outer tubular member 18 to have a smaller thickness of the side wall because each elongate recessed surface 130 can have a smaller thickness or height while enabling generally the same volume of fluid to flow within the interior passages 64.

The formation of interior passages 64 can be more thoroughly understood as follows. After slidably inserting the inner portion 104 within the outer portion 102 to form the outer tubular member 18, the inner portion 104 becomes coaxially disposed within the outer portion 102. With this arrangement, the protrusions 150 contact the inner surface 107 of the outer portion 102, thereby forming the individual interior passages 64 between each of the elongate recessed surfaces 130 and the inner surface 107 of the outer portion 102. Accordingly, in one aspect, each adjacent pair of protrusions 150 defines the side walls of each respective interior passage 64. The interior passages 64 extend a majority of the length (represented by “L” in FIG. 2) of the outer tubular member 18. In one aspect, a surface 141 of circular recess 140 (as seen in FIG. 2) and a bottom portion of each elongate recessed surface 130 have substantially the same elevation at junction 155 (between circular recess 140 and the elongate recessed surfaces 130) to provide a generally seamless transition therebetween.

As briefly indicated above, various systems (e.g., system 5) of the disclosure include fluid source 32, which is provided to lubricate a treatment site and/or to cool the bur 24 (or any other bur of the disclosure). Once the bur 24 is positioned at a treatment site to debride target bone, fluid can be supplied from fluid source 32, which flows from the bottom opening 31 of the irrigation port 30, through the interior passages 64 to irrigate the bur 24 and/or the treatment site after exiting proximate the distal end 45. The illustrated arrangement enables both the direct cooling of an optionally provided bearing assembly 61 (discussed in detail below) as well as flooding the treatment site with fluid, as appropriate to the procedure, while the bur 24 is rotating to cut the target bone or tissue. As with the aspiration pathway, a smaller overall, cross-sectional profile of instrument 10 is maintained in accordance with the smaller cross-sectional profile achieved via providing the irrigation passage(s) 64 within the outer tubular member 18. Because the irrigation interior passages 64 are contained internally within the outer tubular member 18, the outer tubular member 18 has a smaller overall cross-sectional profile. In another aspect, the outer surface 74 of the outer tubular member 18 is generally uniform and generally smooth without significant protrusions, such as the protrusion(s) that would otherwise be formed by an irrigation tube externally attached to instrument as seen in conventional instruments.

With further reference in particular to FIGS. 3A-4B, the instrument 10 can optionally further include a bearing assembly 61 that limits axial movement of the bur 24 (or any other bur disclosed herein) relative to outer tubular member 18, and limits axial movement of the bur 24 in a direction parallel to the central axis A (FIG. 1) of the body 71/distal section 44. In the embodiment illustrated, the bearing assembly 61 includes a plurality of balls 62 (three in the illustrated embodiment) maintained within the outer tubular member 18 and coupled with the bur 24 through the circular race 63 (FIGS. 4A and 5). In particular, the plurality of balls 62 are positioned within corresponding apertures 106 located within the protrusions 150. When fully assembled as shown in FIG. 4B, the balls 62 engage the circular race 63 and are covered by the outer portion 102.

The balls 62, in one embodiment, are spherically shaped and formed of ceramic material equally spaced about the circular race 63 (i.e., spaced about 120° from one another), which is machined into the bur 24 near the proximal end 73 of the bur 24. In other embodiments, a fewer or greater number of balls than three can be used. The bur 24, and in particular the race 63, can be formed of various materials, examples of which include, but are not limited to 440 stainless steel, M2 tool steel, carbide, etc. Thus, in one embodiment, the balls 62 are formed of a first material (e.g., ceramic) and the race 63 is formed of a second material (e.g., 440 stainless steel), different than the first material. In some instances, the use of different materials for balls 62 and race 63 can preclude galling and/or wear.

Regardless of the materials selected for the balls 62 and race 63, the bearing assembly 61 controls an axial and radial position of the bur 24 with respect to the outer portion 102, allowing for precise tracking of the bur 24 while utilized with an electromagnetic image guided system, which will be further discussed below. Additionally, rolling contact between the balls 62 and race 63 provides reduced friction when compared to sliding contact between the bur 24 and inner portion 104. Thus, a temperature of the outer portion 102 during operation is reduced such that damage of tissue or bone proximate and/or in contact with the outer portion 102 can be prevented. Moreover, a separate thrust bearing for instrument 10 is not needed. The bearing assembly 61 can operate and is similar in construction to a ball bearing, wherein the inner portion 104 acts as a ball carrier for balls 62, bur 24/race 63 acting as an inner race. The outer portion 102 need not bear a load from the balls 62 and can be provided so as to hold the balls 62 in place within the apertures 106. Generally, upon final assembly, the bearing assembly 61 restricts axial motion of the bur 24 while allowing rotation of the bur 24 relative to the outer portion 102 and the inner portion 104.

For all of the embodiments disclosed herein, the outer hub 16 can be coupled with a suitable tracking device 39 (schematically depicted in FIG. 1) of the system 5 such that instrument 10 can be used with a suitable electromagnetic image guided system that can determine a position of the bur 24 (or any alternate bur disclosed herein) within a patient's anatomy. In one embodiment, the tracking device 39 includes one or more magnetic coils detectable by the image guided system that can be used to display a representation of the instrument 10 and bur 24 with respect to a patient's anatomy. The magnetic coils can provide a reference location detectable by the image guided system such that, based on the reference location, an orientation and position of the bur 24 can be determined by the image guided system during a pre-operation procedure (e.g., utilizing a computed tomography (CT) or magnetic reasonable imaging (MM) scan). During a surgical procedure, an indication of the orientation and position of bur 24 can be provided to assist a surgeon, for example by providing an image of the bur 24 on a monitor with respect to a patient's anatomy. One exemplary electromagnetic image guided system or tracking device is disclosed in U.S. Pat. No. 8,504,139, filed on Mar. 10, 2009, entitled “Navigating A Surgical Instrument”, the contents of which are hereby incorporated by reference in their entirety.

FIG. 6 illustrates an alternate bur 224 that can be used as a substitute for bur 24. The bur 224 is identical to bur 24 in all ways not expressly stated. In this embodiment, the bur includes an aspiration pathway 275a, 275b, however, the second portion 275b and an opening 276 are provided proximal with respect to a head 270, such as within a body 271 of the bur 224 between the head 270 and a proximal end 273 of the bur 224. The second portion of the aspiration pathway 275b can optionally intersect the first portion of the aspiration pathway 275a proximate a race 263, which can be configured identically to and function in the same way with respect to race 63. In this alternate embodiment, a centerline C′ of the second portion of the aspiration pathway 275b is orientated at an angle ϕ with respect to a central axis A′ of the body 271 and bur 224, which is also a centerline of the first portion of the aspiration pathway 275a, however, this need not be the case in alternate embodiments. The angle ϕ can be in the range of about 10 to about 90 degrees with respect to the central axis A′. In one particular embodiment, the angle ϕ is about 24 degrees.

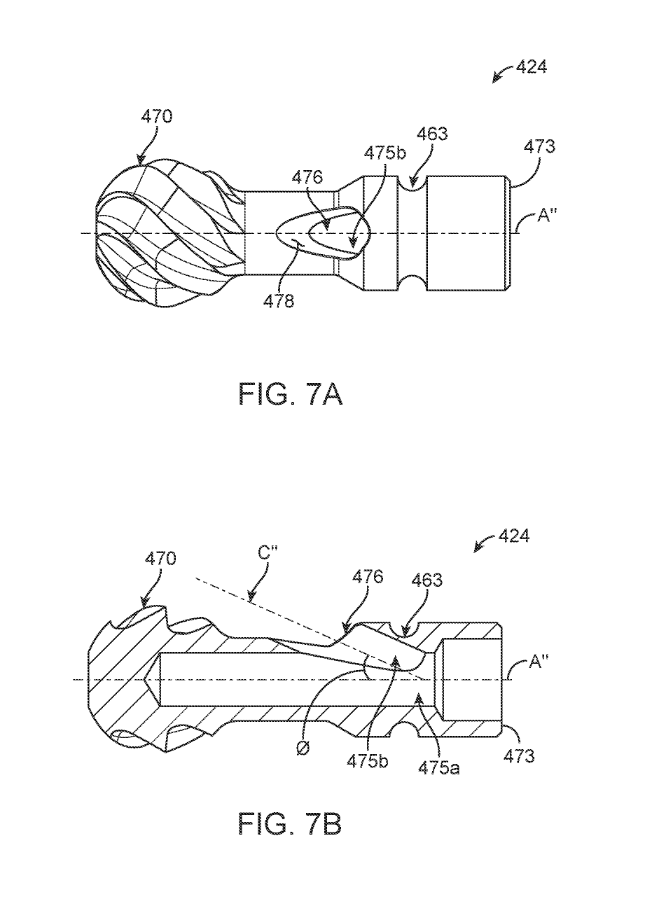

FIGS. 7A-7B illustrate yet another surgical bur 424 that can be used with the system 5 and the surgical instrument 10 by attaching a proximal end 473 of the bur 424 to the distal section 145 of the inner tubular member 22 as described above with respect to other embodiments. Generally, the bur 424 is an alternate to the burs 24, 224 disclosed herein. The bur 424 is identical to bur 24 in all ways not expressly stated. The bur 424 includes a head 470, a body 471 and optionally a race 463 for a bearing assembly 61 (it will be understood that race 463 can be identical to race 63). In the illustrated embodiment, the head 470 has a fluted configuration, however, any of the aforementioned head configurations are also envisioned. As with prior embodiments, an aspiration pathway 475a, 475b is provided within the body 471. The aspiration pathway includes a first portion 475a and a second portion 475b. The second portion of the aspiration pathway 475b is provided proximal to the head 470 and has an opening 476 leading out of the body 471, similar to the embodiment of FIG. 6. The aspiration pathway 475 can optionally include a ramped surface 478 (FIG. 7A) proximate and extending into the body 471 from the opening 476. In this alternate embodiment, proximate the opening 476, the second portion of the aspiration pathway 475b is configured to define a centerline C″ that is orientated at an angle ϕ of about 24 degrees with respect to a central axis A″ of the body 471 and bur 424. It is noted that in this illustrated embodiment, the central axis A″ is the same as a centerline of the first portion of the aspiration pathway 475a, however, this need not be the case. The angle ϕ can be in the range of about 10 to about 90 degrees with respect to the central axis A″.

Regardless of exact form, the systems, micro-burring instruments and burs of the present disclosure are useful in performing various sinus operations and other procedures. By way of example, in a surgical procedure, the instrument 10 is maneuvered to the treatment site and the bur 24 (or any alternate bur disclosed herein) is positioned against the bone or other target tissue. Next, the inner tubular member 22 is then rotated relative to the outer tubular member 18, such that the bur 24 burs (e.g., cuts or abrades) the contacted cartilage and/or bone. The treatment site can be continuously flushed with an irrigation fluid via the interior passages 64. Other related surgical techniques may be performed before, during, or after application of instrument (e.g., instrument 10).

In addition to the surgical procedure generally outlined above, the systems, micro-burring instruments and burs of the present disclosure can be used to perform a variety of other surgical procedures in which hard tissue or bone is debrided or cut while flooding the treatment site with fluid to irrigate the bur and the target tissue or bone.

### Example

The present inventors conducted an experiment to document the propensity for clogging of burs encompassed by the present disclosure (e.g., burs 24, 224). Previous testing was performed to characterize the performance of a standard 30K reverse taper diamond bur model number 1884015RTD (“standard 30K bur”) available from Medtronic Inc. of Minneapolis, Minn. Similarly, the surgical burs 24, and 224 were prototyped and incorporated into the system 5 and the surgical instrument 10 in an effort to evaluate a propensity for clogging when compared to the standard 30K bur. Both in the previous testing of the standard 30K bur, and this testing, a ham hock was used as the test media as it is believed to closely simulate the density of the bone found in the clivus.

Two samples of each prototype bur 24 (head suction), 224 (proximal suction) were tested (i.e. used for debriding the ham hock). Sample 3 (a proximal suction design (i.e. bur 224 shown in FIG. 6) clogged early in the test, and the clog could not be cleared. As this test was to characterize clogging, the test was aborted for Sample 3. Clogging results can be found in TABLE 1 and are summarized in Table 2 below.

TABLE 1

| | | Time to | Clog | | Mean | Total |
|---|---|---|---|---|---|---|
| Sample | Description | clog (s) | location | Cleared | Each | Mean |
| 1 | Proximal | 90 | Opening | Yes | 151.25 | 170.625 |
| | Suction | 120 | Opening | Yes | | |
| | | 290 | Opening | Yes | | |
| | | 105 | Opening | Yes | | |
| 2 | Head | N/A | N/A | N/A | | 165 |
| | Suction | | | | | |
| 3 | Proximal | 200 | Opening | | 190 | |
| | Suction | 180 | Opening | No | | |
| | *Permanent | | | | | |
| | Clog | | | | | |
| 4 | Head | 165 | Opening | Yes | 165 | |
| | Suction | | | | | |

TABLE 2

| | Clog/min |
|---|---|
| Head Suction | 0.033333333 |
| Proximal Suction | 0.285714286 |
| Standard 30K | constant |

In the previous testing of the standard 30K burs on a ham hock in the same manner as the current test, the standard 30 k burs suffered almost instantaneous and continuous clogging upon drilling and after each respective clog was cleared and testing resumed. Both of the tested embodiments of the present disclosure (burs 24, 224) offer significant improvement by comparison. The proximal suction bur 224 clogged less frequently as compared to the standard 30K bur but was unable to be de-clogged, when clogging did occur, due to the size/angle of the aspiration passageway in this particular test and the stylet used for clearing clogs. In the present example, the stylet used was part number 66E1139 available from Medtronic Inc. of Minneapolis, Minn. Results from the head suction bur 24 are noticeable improvements over the standard 30K bur as not only was clogging significantly reduced (compared to the proximal suction bur 224 as well as the standard 30K bur), any clogs that did arise were able to be cleared with a stylet.

Although the present disclosure has been described with reference to preferred embodiments, workers skilled in the art will recognize that changes can be made in form and detail without departing from the spirit and scope of the present disclosure.

What is claimed is:

1. A surgical bur comprising:
a body;
a head connected to the body; wherein the head and the body collectively define a central axis; and
an aspiration pathway defining a centerline that is arranged at an angle with respect to the central axis.

2. The surgical bur of claim 1, wherein the head has a spherical shape.

3. The surgical bur of claim 1, wherein the angle is between about 10 and about 40 degrees.

4. The surgical bur of claim 3, wherein the angle is about 18 to about 22 degrees.

5. The surgical bur of claim 1, wherein the aspiration pathway includes a first portion and a second portion and further wherein a centerline of the first portion is angled with respect to a centerline of the second portion.

6. The surgical bur of claim 1, wherein the aspiration pathway terminates at an opening and the aspiration pathway forms a ramped surface proximate the opening.

7. The surgical bur of claim 1, wherein the aspiration pathway defines an opening that extends through the head.

8. The surgical bur of claim 1, wherein the aspiration pathway defines and opening that extends through the body, proximal with respect to the head.

9. A surgical instrument comprising:
an outer tubular member having a proximal section, a distal section, and a central lumen; and
an inner tubular member rotatably received within the central lumen, wherein the inner tubular member includes a lumen and a distal end of the inner tubular member forms a bur extending distally beyond, and exposed relative to, the distal section; wherein the bur includes:
a body;
a head connected to the body; wherein the head and the body collectively define a central axis; and
an aspiration pathway defining a centerline that is arranged at an angle with respect to the central axis; wherein the aspiration pathway is in communication with the lumen of the inner tubular member.

10. The surgical instrument of claim 9, wherein the aspiration pathway includes a first portion and a second portion and further wherein a centerline of the first portion is angled with respect to a centerline of the second portion.

11. The surgical instrument of claim 9, wherein the aspiration pathway terminates at an opening and the aspiration pathway forms a ramped surface proximate the opening.

12. The surgical instrument of claim 9, wherein the head has a spherical shape.

13. The surgical instrument of claim 9, wherein the angle is between about 10 and about 40 degrees.

14. The surgical instrument of claim 13, wherein the angle is about 18 to about 22 degrees.

15. The surgical instrument of claim 9, further including a sleeve within the lumen of the inner tubular member.

16. The surgical instrument of claim 9, wherein the aspiration pathway defines an opening that extends through the head.

17. The surgical instrument of claim 9, wherein the aspiration pathway defines and opening that extends through the body, proximal with respect to the head.

18. The surgical instrument of claim 17, wherein the opening is positioned proximal to the head.

19. The surgical instrument of claim 9, wherein the surgical instrument includes an inner tube defining a plurality of planar sections and further wherein each planar section includes an aperture that receives a ball of a bearing assembly.

20. The surgical instrument of claim 19, wherein the bur defines a race in which the ball is positioned.
